(19) United States
(12) Patent Application Publication
(10) Pub. No.: US 2025/0031623 A1
(43) Pub. Date: Jan. 30, 2025

(54) Corn Detasseling Inspection Robot and Method for Performing Detasseling by Using Same

(71) Applicant: Intelligent Equipment Research Center, Beijing Academy of Agriculture and Forestry Sciences

(72) Inventors: Liping CHEN, Beijing (CN); Ruirui ZHANG, Beijing (CN); Chenchen DING, Beijing (CN); Gang XU, Beijing (CN); Tongchuan YI, Beijing (CN)

(21) Appl. No.: 18/275,184

(22) PCT Filed: May 12, 2023
(86) PCT No.: PCT/CN2023/093869

§ 371 Date: Jul. 31, 2023

(30) Foreign Application Priority Data
Apr. 13, 2023 (CN) 202310390701.X

(51) Int. Cl. A01D 47/00; G01C 9/02; G06V 20/10

(52) U.S. Cl. CPC A01D 47/00; G01C 9/02; G06V 20/188; G06V 2201/06

(57) ABSTRACT

Provided is a corn detasseling inspection robot and a method for performing detasseling by using the same. The corn detasseling inspection robot includes a control terminal, a moving device and a detasseling device; wherein the control terminal includes a detection module, a detasseling control module and a tassel recognition module; the detection module is configured for acquiring an image of a corn plant; the tassel recognition module is configured for judging whether there is a corn tassel on the corn plant based on the image of the corn plant, and determining a position of a root of the corn tassel based on a detasseling operation point prediction model in the case where there is the corn tassel on the corn plant; and the detasseling control module is configured for controlling the moving device and using the detasseling device to perform detasseling based on the position of the root.

### TECHNICAL FIELD

The present disclosure relates to the field of the mechanical arm, and in particular, to a corn detasseling inspection robot and a method for performing detasseling by using the same.

### BACKGROUND

At present, the seed corn detasseling is primarily performed manually, resulting in high labor intensity, low detasseling efficiency and difficulties in ensuring detasseling quality. At the same time, labor costs for the seed corn detasseling are expensive, leading to high seed production costs for seed production enterprise and seriously impacting the development of high-quality seed production industry. Mechanized and intelligentized detasseling which is highly efficient and less affected by natural conditions and human factors, have the advantages of high maneuverability, high intelligence, easy formulation and good operating conditions, etc. Using the mechanized and intelligentized detasseling to replace manual detasseling is an inevitable trend for the corn seed production industry.

Currently, traditional ground detasseling machines are easily affected by factors such as differences in terrain, height of corn plants and planting density. The single detasseling rate is not high, and there are problems such as error and leakage detasseling. After the detasseling is performed using the traditional ground detasseling machines, there is often a need for repeated search and detasseling by either the traditional ground detasseling machine or manual labor.

### SUMMARY

Provided are a corn detasseling inspection robot and a method for performing detasseling using the same, which are used for solving the challenges encountered by conventional detasseling machines, which are prone to be affected by factors like variations in terrain, height of corn plant and planting density which often leading to error and leakage detasseling, resulting in a low single detasseling rate.

In the first aspect, the present disclosure provides a corn detasseling inspection robot, including:
a control terminal, a moving device and a detasseling device;
wherein, the detasseling device is provided on the moving device; the control terminal includes a detection module, a detasseling control module and a tassel recognition module which are electrically connected to one another; the detasseling control module is electrically connected with the moving device and the detasseling device;
the detection module is configured for acquiring an image of a corn plant during the moving device moves in a preset area;
the tassel recognition module is configured for judging whether there is a corn tassel on the corn plant based on the image of the corn plant, and determining a position of a root of the corn tassel based on a detasseling operation point prediction model in the case where there is the corn tassel on the corn plant; and
the detasseling control module is configured for controlling the moving device and using the detasseling device to perform detasseling based on the position of the root.

The corn detasseling inspection robot according to the present disclosure, the detasseling device includes a detasseling module;
wherein, the detasseling module includes a detasseling support, an elastic member, a guide pipe, a pull string, a motor and a detasseling assembly; and
the detasseling support is connected to one side of the moving device; the motor and the guide pipe are connected on the detasseling support; the detasseling assembly is slidably disposed in the guide pipe; the elastic member is fixed in the guide pipe; the detasseling assembly abuts against the elastic member; and one end of the detasseling assembly is connected with a rotation shaft of the motor via the pull string.

The corn detasseling inspection robot according to the present disclosure, the detasseling assembly includes a pellet, the pellet is slidably provided in the guide pipe and abuts against the elastic member, and one end of the pellet is connected to the rotation shaft of the motor via the pull string.

The corn detasseling inspection robot according to the present disclosure, the detasseling assembly includes a knife edge and a knife rod;
the knife edge is connected with the knife rod, the knife edge and the knife rod are slidably provided in the guide pipe, the knife rod abuts against the elastic member, and one end of the knife rod away from the knife edge is connected with the rotation shaft of the motor via the pull string.

The corn detasseling inspection robot according to the present disclosure, a limiting portion is provided on the knife rod, a limiting groove is provided on a side wall of the guide pipe, and the limiting portion is slidably provided in the limiting groove.

The corn detasseling inspection robot according to the present disclosure, the detasseling device further includes a main beam;
at least one side of the main beam is provided with a slid rail, and the detasseling support of the detasseling module is slidably provided on the slid rail.

The corn detasseling inspection robot according to the present disclosure, a gyroscope and a damping module are provided in the moving device, the gyroscope is configured for detecting an inclination degree of the moving device, and the damping module is configured for adjusting an inclination angle of the moving device based on the inclination degree.

In a second aspect, the present disclosure further provides a method. The method for performing detasseling by using the corn detasseling inspection robot as described above, including:
acquiring the image of the corn plant during the moving device moves in the preset area;
judging whether there is the corn tassel on the corn plant based on the image of the corn plant, and determining the position of the root of the corn tassel based on the detasseling operation point prediction model in the case where there is the corn tassel on the corn plant;
controlling the moving device and using the detasseling device to perform detasseling based on the position of the root.

The method according to the present disclosure, before the step of acquiring the image of the corn plant around the moving device, the method includes:
selecting a corn plant sample and acquiring a standard image of the corn plant sample;
establishing the detasseling operation point prediction model based on the standard image of the corn plant sample.

The method according to the present disclosure, the step of controlling the moving device and using the detasseling device to perform detasseling based on the position of the root includes:
controlling the moving device to stop, acquiring a profile information of a stem of the corn plant, and extracting the position of the root of the corn tassel;
hitting the root of the corn tassel by using the detasseling device based on the position of the root;
determining a detasseling result of a current corn tassel based on a result of hitting the root of the corn tassel.

The corn detasseling inspection robot in this disclosure is equipped with a moving device that enables automatic inspection within the designated area, and recognizes and treats the corn tassel of the corn plant in the preset area; at the same time, utilizing the detection module and the tassel recognition module, based on the detasseling operation point prediction model, the image of the corn plant is collected, and the corn tassel is recognized and positioned, the position of the root of the corn tassel can be positioned more accurately without being affected by the differences in terrain and height of the corn plant, so that the detasseling device can align with the root of the corn tassel and perform detasseling, thereby improving the detasseling accuracy rate and the success rate, avoiding situations such as error detasseling and leakage detasseling, and effectively solving the problem that the conventional detasseling machines are prone to be affected by factors like variations in terrain, height of corn plant and planting density which leading to situations such as error and leakage detasseling, resulting in a low single detasseling rate.

### BRIEF DESCRIPTION OF THE DRAWINGS

To describe the technical solutions in the embodiments of the present disclosure or in the prior art more clearly, the following briefly introduces the accompanying drawings required for describing the embodiments or the prior art. Apparently, the accompanying drawings in the following description show merely some embodiments of the present disclosure, and a person of ordinary skill in the art may still derive other drawings from these accompanying drawings without creative efforts.

FIG. 1 is a schematic diagram of a corn detasseling inspection robot provided in an embodiment of the present disclosure;

FIG. 2 is a schematic diagram of a system of the corn detasseling inspection robot provided in the embodiment of the present disclosure;

FIG. 3 is a schematic diagram of a detasseling module provided in the embodiment of the present disclosure;

FIG. 4 is a sectional view of the detasseling module provided in the embodiment of the present disclosure;

FIG. 5 is a schematic diagram of a detasseling module provided in another embodiment of the present disclosure;

FIG. 6 is a schematic flowchart of a method for performing detasseling by using a corn detasseling inspection robot provided in an embodiment of the present disclosure;

FIG. 7 is a second schematic flowchart of a method for performing detasseling by using acorn detasseling inspection robot provided in an embodiment of the present disclosure;

FIG. 8 is a schematic diagram of determining a position of a root of a corn tassel of a corn plant provided in an embodiment of the present disclosure;

FIG. 9 is a third schematic flowchart of a method for performing detasseling by using a corn detasseling inspection robot provided in an embodiment of the present disclosure;

FIG. 10 is a schematic diagram of determining a position of a root of a corn tassel of a sample corn plant provided in an embodiment of the present disclosure; and

FIG. 11 is a fourth schematic flowchart of a method for performing detasseling by using a corn detasseling inspection robot provided in the embodiment of the present disclosure;

Reference signs:
1, corn detasseling inspection robot;
11, control terminal; 12, moving device; 13, detasseling device;
111, detection module; 112, detasseling control module; 113, tassel recognition module; 131, detasseling support; 132, elastic member; 133, guide pipe; 134, pull string; 135, motor; 136, detasseling assembly; 137, main beam;
1331, limiting groove; 1361, pellet; 1362, knife edge; 1363, knife rod; 1364, limiting portion; 1371, slide rail.

### DETAILED DESCRIPTION OF THE EMBODIMENTS

To make the objectives, technical solutions, and advantages of the present disclosure clearer, the following section clearly and completely describes the technical solutions in this disclosure with reference to the accompanying drawings in the present disclosure. Apparently, the embodiments to be described are merely a part rather than all of the embodiments of the present disclosure. All other embodiments obtained by a person of ordinary skill in the art based on the embodiments of the present disclosure without creative efforts shall belong to the scope of the present disclosure.

In the description of the embodiments of the present disclosure, it is important to note that, orientation or position relationships indicated by terms such as “up”, “down”, “left”, “right”, “inside” and “outside” are based on orientation or position relationships shown in the accompanying drawings, These terms are employed for convenience and simplicity of describing the embodiments and should not be interpreted as implying any fixed orientation or construction requirement for the indicated device or element. Therefore, it should be understood that the embodiments of the present disclosure are not limited by these orientation or position terms.

In the description of the present disclosure, it is important to note that the terms the terms “mounted” and “connected”, unless specified or limited otherwise, should be interpreted in a broad sense. For example, they can refer to fixed connections, detachable connections, or integral connections; they may also be mechanical or electrical connections; may also be direct connections or indirect connections via intervening structures; and may also be inner communications of two elements. The specific meanings of the above terms in the present disclosure can be understood by those skilled in the art according to specific situations.

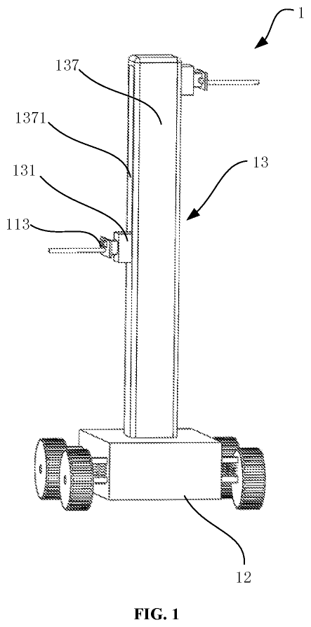

The following describes a corn detasseling inspection robot provided in the present disclosure with reference to FIG. 1 to FIG. 5.

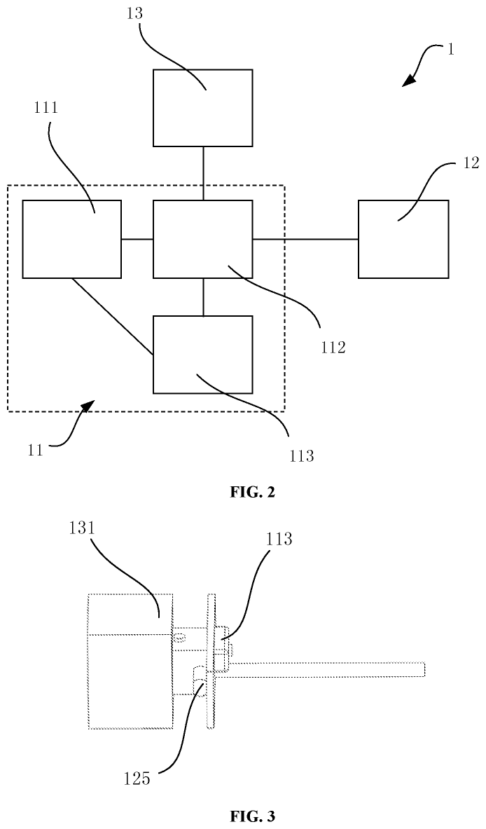

As shown in FIGS. 1 and 2, the corn detasseling inspection robot 1 of the present disclosure includes: a control terminal 11, a moving device 12 and a detasseling device 13; the detasseling device 13 is arranged on the moving device 12; the control terminal 11 includes a detection module 111, a detasseling control module 112 and a tassel recognition module 113 which are electrically connected to one another; the detasseling control module 112 is electrically connected to the moving device 12 and the detasseling device 13; the detection module 111 is configured for acquiring an image of a corn plant during the moving device 12 moves in a preset area; the tassel recognition module 113 is configured for judging whether there is a corn tassel on the corn plant based on the image of the corn plant, and determine a position of a root of the corn tassel based on a detasseling operation point prediction model in the case where there is the corn tassel on the corn plant; the detasseling control module 112 is configured for controlling the moving device 12 and using the detasseling device 13 to perform detasseling based on the position of the root.

In this embodiment, the corn detasseling inspection robot 1 is generally used for performing a detasseling treatment on a corn tassel of a corn plant in a corn field, which can not only complete the inspection and detasseling of the whole corn field independently, but also perform detasseling of the remaining corn tassel on the corn field after large-area detasseling is performed by using a traditional machine or artificial detasseling. The moving device 12 can automatically drive the corn detasseling inspection robot 1 to move along a preset route in a preset area and approach various corn plants in the preset area so as to recognize and treat the corn tassels; the preset area is usually a cornfield area in which corn detasseling is needed to be performed; the detection module 111 can acquire image information of a corn plant by means of shooting, scanning, etc. during the movement of the moving device 12, and transmits the corresponding image information to the tassel recognition module 113; by providing the tassel recognition module 113, the image of the corn plant is processed and analyzed, and the position of the root of the corn tassel can be positioned more accurately based on the detasseling operation point prediction model, so that the detasseling device 13 can perform detasseling; after receiving the information of the position of the root of the corn tassel, the detasseling control module 112 controls the moving device 12 to automatically perform fine adjustment and enable the detasseling device 13 to approach and align with the root of the corn tassel, so that the detasseling device 13 removes the corn tassel in a manner such as striking or cutting.

The corn detasseling inspection robot 1 of the present disclosure are driven to automatically perform inspection in the preset area by the moving device 12 arranged, and recognizes and treats the corn tassel of the corn plant in the preset area; at the same time, by means of the detection module 111 and the tassel recognition module 113, based on the detasseling operation point prediction model, the image of the corn plant is collected, and the corn tassel is recognized and positioned, the position of the root of the corn tassel can be positioned more accurately without being affected by the differences in terrain and height of the corn plant, so that the detasseling device 13 can align with the root of the corn tassel and perform detasseling, thereby improving the detasseling accuracy rate and the success rate, avoiding situations such as error and leakage detasseling, and effectively solving the problem that the conventional detasseling machines are prone to be affected by factors like variations in terrain, height of corn plant and planting density which leading to situations such as error and leakage detasseling, resulting in a low single detasseling rate.

Specifically, the moving device 12 may include a wheeled unmanned vehicle or a crawl unmanned vehicle, so as to move in a preset area more flexibly and smoothly, and can also better adapt to different terrain.

Optionally, the detasseling device 13 is a mechanical arm mounted on the moving device 12, one end of the mechanical arm is mounted with a cutter for cutting the corn tassel; the mechanical arm can approach and align with the corn tassel under the control of the detasseling control module 112 to remove the corn tassel.

Optionally, the detection module 111 may include one of a binocular camera, an infrared camera, a spectrum camera, and a laser radar or combination thereof. The infrared camera can be used for acquiring a contour image of a corn plant in the case of insufficient lighting, such as at night, so that the corn detasseling inspection robot 1 can inspect at day and night which is beneficial to timely detasseling with regard to the short period of the seed corn detasseling, reduces the degree of labor, and reduces the labor cost.

Optionally, the control terminal 11 can further include a data processing module. The data processing module can record the inspection and detasseling process of the corn detasseling inspection robot 1, compare and analyze images of the corn plant before and after detasseling, so as to determine the detasseling result, and adjust and improve the corn detasseling inspection robot 1 based on the detasseling process and the result record.

Specifically, as shown in FIG. 3 to FIG. 5, the detasseling device 13 includes a detasseling module, and the detasseling module includes a detasseling support 131, an elastic member 132, a guide pipe 133, a pull string 134, a motor 135, and a detasseling assembly 136; the detasseling support 131 is connected to one side of the moving device 12; the motor 135 and the guide pipe 133 are connected on the detasseling support 131; the detasseling assembly 136 is slidably disposed in the guide pipe 133; the elastic member 132 is fixed in the guide pipe 133; the detasseling assembly 136 abuts against the elastic member 132; and one end of the detasseling assembly 136 is connected to a rotation shaft of the motor 135 via the pull string 134.

In the present embodiment, the detasseling support 131 is configured for supporting and fixing other components of the detasseling module; the detasseling support 131 is movably connected to the moving device 12, and can adjust the position under the drive of the moving device 12 so that the guide pipe 133 can be aligned with the corn tassel so as to perform the detasseling; the guide pipe 133 can guide the detasseling assembly 136, the detasseling assembly 136 can slide in the direction of the guide pipe 133 and emerge from the guide pipe 133, move in the direction in which the guide pipe 133 are aligned, and finally hit the corn tassel to realize the detasseling; by arranging the elastic member 132, the pull string 134 and the motor 135 to cooperate with each other, the transmission power is provided for the detasseling assembly 136. Before performing detasseling, the motor 135 rotates to drive the pull string 134 to pull the detasseling assembly 136 to compress the elastic element 132; when the detasseling assembly 136 reaches the preset position, the motor 135 rotates reversely and quickly to release the pull string 134 and the detasseling assembly 136, and the detasseling assembly 136 moves along the guide pipe 133 under the push of the elastic member 132, and is ejected from the opening of the guide pipe, and hit the root of the corn tassel to realize the detasseling; the motor 135 then rotates reversely again, drive the pull string 134 to pull and withdraw the detasseling assembly 136 to reset the detasseling assembly 136 so as to perform a next detasseling, so that the detasseling assembly 136 can be repeatedly ejected to perform the detasseling, and the structure is simple and the cost is saved.

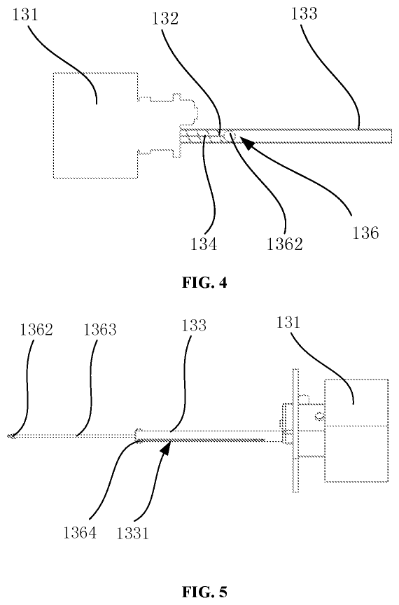

In a specific embodiment, as shown in FIG. 4, the elastic member 132 is a spring, one end of the spring is fixed in the guide pipe 133, and the pull string 134 penetrates through the spring and is connected to one end of the detasseling assembly 136.

Optionally, as shown in FIG. 4, the detasseling assembly 136 includes a pellet 1361. The pellet 1361 is slidably disposed in the guide pipe 133, the pellet 1361 abuts against the elastic member 132, and one end of the pellet 1361 is connected to the rotation shaft of the motor 135 via the pull string 134.

In this embodiment, when the pellet 1361 is withdrawn after being ejected, such arrangement can effectively prevent the pellet 1361 from being caught by an obstacle, and being caught due to an improper attitude when passing through the opening of the guide pipe 133, resulting a withdraw failure, and the structure is simple and more reliable.

In another embodiments, as shown in FIG. 5, the detasseling assembly 136 includes a knife edge 1362 and a knife rod 1363, the knife edge 1362 is connected to the knife rod 1363, the knife edge 1362 and the knife rod 1363 can be slidably disposed in the guide pipe 133, the knife rod 1363 abuts against the elastic member 132, and one end of the knife rod 1363 away from the knife edge 1362 is connected to the rotation shaft of the motor 135 via the pull string 134.

In this embodiment, when performing detasseling, the knife rod 1363 is pushed by the elastic member 132 to eject out from the guide pipe 133, so that the corn tassel can be quickly cut off, and the success rate of detasseling is high.

Specifically, as shown in FIG. 5, the knife rod 1363 is provided with a limiting portion 1364 (barb), the side wall of the guide pipe 133 is provided with a limiting groove 1331, and the limiting portion 1364 is slidably disposed in the limiting groove 1331.

In this embodiment, the limiting portion 1364 and the limiting groove 1331 cooperate with each other to provide a guide and limiting function to the knife rod 1363, so that when the knife rod 1363 is ejected outwards from the guide pipe 133, the knife rod 1363 is kept along the axial direction of the guide pipe 133 without inclination, thereby ensuring that the knife edge 1362 can accurately hit the detasseling position; at the same time, the knife rod 1363 is limited by the limiting groove 1331 and the limiting portion 1364, so that it cannot completely separated from the guide pipe 133, which facilitates the pulling of the knife rod 1363 by the pull string 134 for withdrawing after the detasseling is completed, and the structure is simple and reliable.

In some embodiments not shown in the accompanying drawings, the limiting portion 1364 may also protrude from the inner wall of the guide pipe 133; accordingly, the limiting groove 1331 is arranged on the side wall of the knife rod 1363.

In some embodiments, as shown in FIG. 1, the detasseling device 13 further includes a main beam 137. At least one side of the main beam 137 is provided with a slid rail 1371. The detasseling support 131 of the detasseling module is slidably disposed on the slid rail 1371.

In this embodiment, the slid rail 1371 is generally disposed along the height direction, a slid block is disposed on the detasseling support 131, and the slid block is slidably embedded in the slid rail 1371, so that the detasseling support 131 can slide up and down on the main beam 137, so as to adjust the height of the guide pipe 133, so that the guide pipe 133 can align with the corn tassel, so as to perform the detasseling.

Optionally, as shown in FIG. 1, either side of the main beam 137 can be disposed with the slid rail 1371. Accordingly, two sets of detasseling supports 131 of the detasseling device 13 are disposed on the slid rails 1371 on both sides of the main beam 137 respectively, so that the corn detasseling inspection robot 1 can perform detasseling on corn plants on both sides at the same time without adjusting the detasseling direction, thereby effectively improving the detasseling efficiency; at the same time, by providing the slid rails 1371 and the detasseling supports 131 on both sides of the main beam 137, the mass distribution on both sides of the main beam 137 is more balanced, and the corn detasseling inspection robot 1 is smoother and more reliable in operation.

In some embodiments, a gyroscope and a damping module are provided in the moving device 12. The gyroscope is configured for detecting the inclination degree of the moving device 12, and the damping module is configured for adjusting the inclination angle of the moving device 12 based on the inclination degree.

In this embodiment, by providing the gyroscope and the damping module, the inclination degree of the moving device 12 can be detected and the posture of the moving device 12 can be adjusted, so as to ensure that the detasseling device 13 is always perpendicular to the ground, and facilitate the detasseling control module 112 to control the moving device 12 to use the detasseling device 13 to align with the corn tassel.

Optionally, the damping module may include a damping spring.

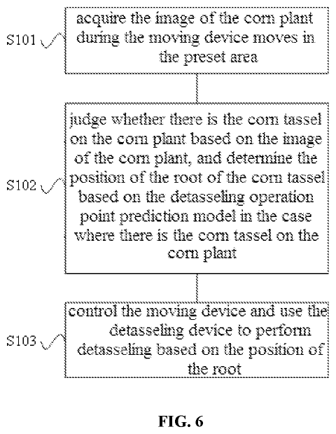

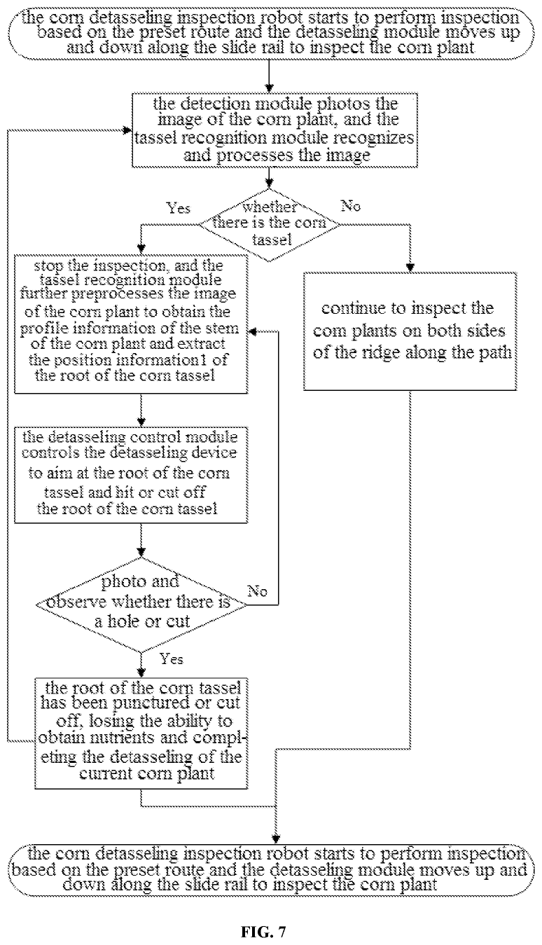

On the other hand, as shown in FIGS. 6-11, the present disclosure further provides a method performing detasseling by using the corn detasseling inspection robot 1 provided by any embodiment of the described embodiments above; the method of the present disclosure using the corn detasseling inspection robot 1 of the described embodiments above also has the advantages of the corn detasseling inspection robot 1 described above, which will not be repeated herein. As shown in FIGS. 6 and 7, the method of the present disclosure includes the following steps:

Step S101: acquiring the image of the corn plant during the moving device moves in the preset area.

Firstly, setting a preset area and a moving route of the moving device 12 based on remote sensing data of a corn planting arrangement in a corn field, so that the moving device 12 approach to the corn plant as possible during the movement; when the corn detasseling inspection robot 1 performs inspection and detasseling in the preset area, the image of the corn plant is obtained by photographing or scanning the corn plant by using an image acquisition equipment such as the binocular camera or the laser radar on the detection module 111.

Specifically, the image acquisition equipment, such as the binocular camera or the laser radar, may be provided on the detasseling support 131, and moves up and down along with the detasseling support 131 on the slid rail 1371. The binocular camera or the laser radar continuously takes pictures or scans to acquire the image of the corn plant.

Step S102: judging whether there is the corn tassel on the corn plant based on the image of the corn plant, and determining the position of the root of the corn tassel based on the detasseling operation point prediction model in the case where there is the corn tassel on the corn plant.

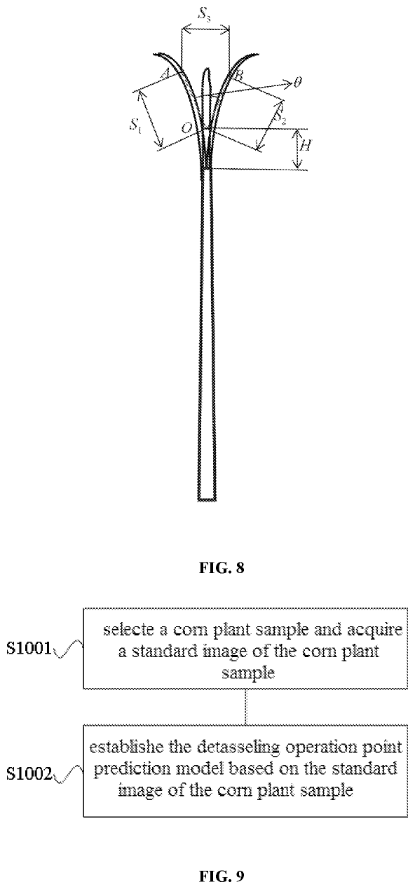

After obtaining the image of the corn plant, the detection module 111 sends the image of the corn plant to the tassel recognition module 113, and the tassel recognition module 113 recognizes the image of the corn plant to determine whether there is the corn tassel on the detected corn plant; if there is not the corn tassel on the corn plant, the corn detasseling inspection robot 1 continues to perform inspection along the moving route; if the corn tassel is recognized, the tassel recognition module 113 further performs operations such as preprocessing and data enhancement on the image of the corn plant, and as shown in FIG. 8, acquiring a position of a point O where the corn tassel and the leaves on both side thereof intersected, a position of a central point A of the curved surface of the leaf on the left side of the corn tassel and a position of a central point B of the curved surface of the leaf, on the right side of the corn tassel and generating a distance S1 between point A and point O, a distance S2 between point B and point O, a distance S3 between point A and point B, and an angle θ of the angle ∠AOB, based on the detasseling operation point prediction model, inputting values of S1, S2, S3 and θ into the model, and acquiring a distance H from an point O to the detasseling point of the corn tassel, the position of the root of the corn tassel is determined based on the value of the distance H.

Step S103: controlling the moving device and using the detasseling device to perform detasseling based on the position of the root.

After the position of the root is acquired, the tassel recognition module 113 sends corresponding information of the position of the root to the detasseling control module 112; the detasseling control module 112 controls the moving device 12 to move and enables the detasseling device 13 to approach and align with the position of the root based on the information of the position of the root; the root of the corn tassel is damaged by means of striking, cutting, etc. in the described embodiments above, so as to achieve detasseling; after detasseling is completed, the detasseling device 13 is reset to an initial state, and the corn detasseling inspection robot 1 continues to perform the inspection and detasseling along the preset route.

In some embodiments, as shown in FIG. 9, before the image of the corn plant is acquired when the moving device moves in the preset area in step S101, the following steps are included:

Step S1001: selecting a corn plant sample and acquiring a standard image of the corn plant sample.

Step S1002: establishing the detasseling operation point prediction model based on the standard image of the corn plant sample.

Firstly, before the corn detasseling inspection robot 1 enters the corn field to perform detasseling, a certain number of corn plants of a certain variety need to be randomly selected from the corn field as samples.

Then, the corn detasseling inspection robot 1 acquires the images of the corn plants by photographing or scanning sides of the corn plant samples by using the image acquisition equipment such as the binocular camera or the laser radar on the detection module 111.

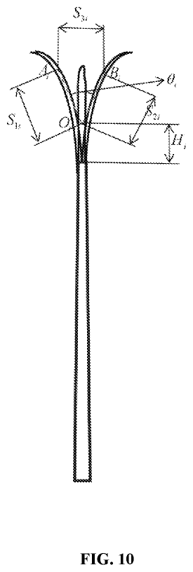

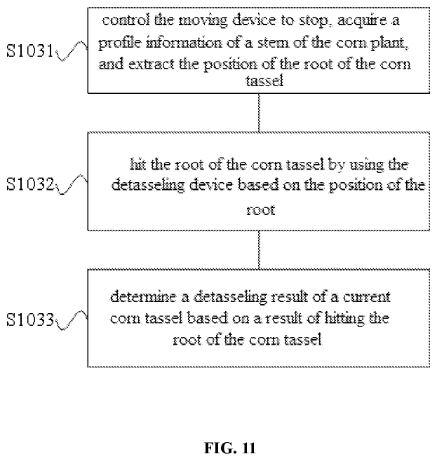

After acquiring the images of the corn plants, the detection module 111 sends the image of the corn plant to the tassel recognition module 113, the tassel recognition module 113 further performs operations such as preprocessing and data enhancement on the images of the corn plants, as shown in FIG. 10, acquiring a position of a point Oi where the corn tassel of a corn plant sample i and leaves on both sides thereof are intersected, a position of a central point Ai of the curved surface of the leaf on the left and a position of a central point Bi of the curved surface of the leaf on the right, and then, generating a distance S1i between the point Oi and the point Ai, a distance S2i between the point Oi and the point Bi, a distance S3i between the point Ai and the point Bi, and an angle θi of the angle ∠AiOiBi.

Then, the corn tassel of the corn plant sample is manually removed, the distance Hi from the point Oi to the root of the corn tassel is measured, and the distance Hi is input into the corn detasseling inspection robot 1.

Finally, the corn detasseling inspection robot 1 processes and analyses the data S1i, S2i, S3i, Oi and Hi, and generates the detasseling operation point prediction model:

F(S1,S2,S3,θ)=H

Based on the detasseling operation point prediction model, a corresponding geometric parameter of a corn plant to be detasseled can be input into the detasseling operation point prediction model when performing detasseling. In this way, the distance H from an point O where the corn tassel and leaves on both sides thereof are intersected to the root of the corn tassel can be acquired, so as to pre-position a position of a root of acorn tassel of an actual corn plant, so as to assist the detasseling control module 112 controls the detasseling device 13 to align with the root of the corn tassel, improving the efficiency and success rate of detasseling.

In some embodiments, as shown in FIGS. 7 and 11, step S103 of the moving device is controlled and the detasseling device is used to perform detasseling based on the position of the root includes the following steps:

Step S1031: controlling the moving device to stop, acquiring a profile information of a stem of the corn plant, and extracting the position of the root of the corn tassel.

Step S1032: hitting the root of the corn tassel by using the detasseling device based on the position of the root.

Step S1033: determining a detasseling result of a current corn tassel based on a result of hitting the root of the corn tassel.

In this embodiment, the image recognition system such as the laser radar or the vision system is provided on the detasseling device 13. After acquiring the position of the root, the tassel recognition module 113 sends the corresponding information of the position of the root to the detasseling control module 112. The detasseling control module 112 controls the moving device 12 to stop. The image recognition system on the detasseling device 13 further scans and recognizes the profile of the stem of the corn plant based on the position of the root provided by the detasseling operation point prediction model, thus, the root of the actual corn tassel of the corn plant to be detasseled currently are captured, so that the acquired root position is more accurate, which is beneficial to improve the success rate of the detasseling.

Then, the detasseling control module 112 controls the moving device 12 to move and drive the detasseling device 13 approach and align with the position of the root based on the information of the position of the root, and the detasseling is realized by means of damage to the root of the corn tassel in manners such as striking or cutting in the described embodiments above.

After a single detasseling is completed, the detection module 111 again performs photographing and recognition on the corn tassel of the current corn plant, if the root of the corn tassel is not completely broken down or cut off, controlling the detasseling device 13 to perform detasseling again on the root of the corn tassel. The corn tassel is photographed and recognized again until the detection module 111 can recognize that the root of the corn tassel has been completely broken down or cut off, namely, the detasseling of the corn plant is completed, and then the inspection is performed continually along the preset route until the inspection and detasseling of the entire preset area is completed. In the present embodiment, the detection module 111 repeatedly performs photographing, recognition and determination on the detasseling result, and the detasseling is performed repeatedly on the corn tassel which has not been completely detasseled, thereby avoiding the situation of leakage detasseling and incomplete detasseling, and improving the efficiency and accuracy of detasseling.

In some embodiments, when inspection and detasseling are performed in the case of insufficient light, such as at night, the corn detasseling inspection robot 1 photos the corn plant by means of the infrared camera of the detection module 111, and determines, based on the profile information of the corn plant, whether there a corn tassel on the corn plant; if there is acorn tassel on a current corn plant, a geometric parameter of the corn plant is measured based on profile information of the corn plant, a specific measurement point is as described in the above embodiments, and details are not described herein again; once the geometric parameter is acquired, the position of the root of the corn tassel can be determined based on the detasseling operation point prediction model, the moving device 12 is controlled to move and the detasseling device 13 is driven to approach and align with the position of the root based on the information of the position of the root, and the root of the corn tassel is damaged by means of striking, cutting, etc. in the described embodiments above, so as to achieve detasseling; after the detasseling is completed, the corn plant is photographed again by the infrared camera, and whether the corn tassel is completely removed is judged by judging whether there is a hole profile at the position of the root of the corn tassel in the acquired corn plant profile or whether the corn tassel has been cut off, so as to judge whether the detasseling of the current corn plant is completed; if the detasseling is not completed, the root of the corn tassel need to be positioned and detasseled again, until the detection module 111 can recognize that the root of the corn tassel have been completely broken down or cut off.

Optionally, during inspection of the corn detasseling inspection robot 1, a data processing module may be set to record the whole inspection and detasseling process, and record the position of each corn plant, pictures before and after detasseling, and the consumed time of detasseling of each corn plant, so that the user can determine whether the corn plant is completely detasseled, and based on this, the inspection route and the running mode of the corn detasseling inspection robot 1 are adjusted, improving the efficiency and accuracy of detasseling.

For example, after a traditional detasseling machine or artificial detasseling is used to perform large-area detasseling, the corn detasseling inspection robot 1 may be used to perform detasseling of the reminding corn tassel; after the detasseling and inspection of the reminding corn tassel of the whole seed corn field is completed, based on the position and number N of the reminding corn plant with corn tassel recorded and counted by the data processing module; in conjunction with the total number M of the corn plants in the seed corn field by means of the formula:

P=N/M
the omissions P of the traditional detasseling machine or artificial detasseling performing large-area detasseling of the seed corn field is calculated, so as to determine the effect of the traditional detasseling machine or artificial detasseling, providing a reference for calculating the labor cost of the traditional machinery or artificial large-area detasseling and guiding the optimization and adjustment of the structure of the traditional detasseling machine or the operation flow of artificial detasseling.

Secondly, a user can determine the detasseling operation condition of the corn detasseling inspection robot 1 by means of a difference between images of a corn plant before and after detasseling. Specifically, on the third day after the whole inspection task of the corn field is completed, the corn detasseling inspection robot 1 move again along the inspection route; the spectral camera in a detection module 111 is used to photo the detasseled corn tassel; the activity of the corn tassel is determined based on the water content of the corn tassel, so as to determine the detasseling effect; and the structure, the inspection route and the running mode of the corn detasseling inspection robot 1 are optimized and adjusted based on the detasseling effect.

In addition, the user can calculate, based on the recorded content, the average time consumed by the detasseling of a single reminding corn plan, confirm the operation efficiency of the detasseling inspection robot 1, and optimize and adjust the structure, inspection route and running mode of the corn detasseling inspection robot 1 based on practical requirements.

The embodiments described above are merely exemplary, and some or all of the modules may be selected based on practical requirements to realize the objectives of the solutions of the embodiments. A person of ordinary skill in the art would have been able to understand and implement same without any inventive effort.

Finally, it should be noted that the foregoing embodiments are merely intended for describing the technical solutions of the present disclosure, but not intended to limit the present disclosure; although the present disclosure is described in detail with reference to the foregoing embodiments, a person of ordinary skill in the art should understand that they may still make modifications to the technical solutions described in the foregoing embodiments or make equivalent replacements to some technical features thereof; these modifications or replacements do not make the essence of the corresponding technical solutions depart from the spirit and scope of the technical solutions of the embodiments of the present disclosure.

1. A corn detasseling inspection robot, comprising:
a control terminal, a moving device and a detasseling device;
wherein, the detasseling device is provided on the moving device; the control terminal comprises a detection module, a detasseling control module and a tassel recognition module which are electrically connected to one another; the detasseling control module is electrically connected with the moving device and the detasseling device;
the detection module is configured for acquiring an image of a corn plant during the moving device moves in a preset area;
the tassel recognition module is configured for judging whether there is a corn tassel on the corn plant based on the image of the corn plant, and determining a position of a root of the corn tassel based on a detasseling operation point prediction model in the case where there is the corn tassel on the corn plant; and
the detasseling control module is configured for controlling the moving device and using the detasseling device to perform detasseling based on the position of the root.

2. The corn detasseling inspection robot according to claim 1, wherein the detasseling device comprises a detasseling module;
wherein, the detasseling module comprises a detasseling support, an elastic member, a guide pipe, a pull string, a motor and a detasseling assembly; and
the detasseling support is connected to one side of the moving device; the motor and the guide pipe are connected on the detasseling support; the detasseling assembly is slidably disposed in the guide pipe; the elastic member is fixed in the guide pipe; the detasseling assembly abuts against the elastic member; and one end of the detasseling assembly is connected with a rotation shaft of the motor via the pull string.

3. The corn detasseling inspection robot according to claim 2, wherein the detasseling assembly comprises a pellet, the pellet is slidably provided in the guide pipe and abuts against the elastic member, and one end of the pellet is connected to the rotation shaft of the motor via the pull string.

4. The corn detasseling inspection robot according to claim 2, wherein the detasseling assembly comprises a knife edge and a knife rod;
the knife edge is connected with the knife rod, the knife edge and the knife rod are slidably provided in the guide pipe, the knife rod abuts against the elastic member, and one end of the knife rod away from the knife edge is connected with the rotation shaft of the motor via the pull string.

5. The corn detasseling inspection robot according to claim 4, wherein a limiting portion is provided on the knife rod, a limiting groove is provided on a side wall of the guide pipe, and the limiting portion is slidably provided in the limiting groove.

6. The corn detasseling inspection robot according to claim 2, wherein the detasseling device further comprises a main beam;
at least one side of the main beam is provided with a slid rail, and the detasseling support of the detasseling module is slidably provided on the slid rail.

7. The corn detasseling inspection robot according to claim 2, wherein a gyroscope and a damping module are provided in the moving device, the gyroscope is configured for detecting an inclination degree of the moving device, and the damping module is configured for adjusting an inclination angle of the moving device based on the inclination degree.

8. A method for performing detasseling by using the corn detasseling inspection robot as claimed in claim 1, comprising:
acquiring the image of the corn plant during the moving device moves in the preset area;
judging whether there is the corn tassel on the corn plant based on the image of the corn plant, and determining the position of the root of the corn tassel based on the detasseling operation point prediction model in the case where there is the corn tassel on the corn plant;
controlling the moving device and using the detasseling device to perform detasseling based on the position of the root.

9. The method according to claim 8, wherein before the step of acquiring the image of the corn plant around the moving device, the method comprises:
selecting a corn plant sample and acquiring a standard image of the corn plant sample;
establishing the detasseling operation point prediction model based on the standard image of the corn plant sample.

10. The method according to claim 8, wherein the step of controlling the moving device and using the detasseling device to perform detasseling based on the position of the root comprises:
controlling the moving device to stop, acquiring a profile information of a stem of the corn plant, and extracting the position of the root of the corn tassel;
hitting the root of the corn tassel by using the detasseling device based on the position of the root;
determining a detasseling result of a current corn tassel based on a result of hitting the root of the corn tassel.

11. The method according to claim 8, wherein the detasseling device comprises a detasseling module;
wherein, the detasseling module comprises a detasseling support, an elastic member, a guide pipe, a pull string, a motor and a detasseling assembly; and
the detasseling support is connected to one side of the moving device; the motor and the guide pipe are connected on the detasseling support; the detasseling assembly is slidably disposed in the guide pipe; the elastic member is fixed in the guide pipe; the detasseling assembly abuts against the elastic member; and one end of the detasseling assembly is connected with a rotation shaft of the motor via the pull string.

12. The method according to claim 11, wherein the detasseling assembly comprises a pellet, the pellet is slidably provided in the guide pipe and abuts against the elastic member, and one end of the pellet is connected to the rotation shaft of the motor via the pull string.

13. The method according to claim 11, wherein the detasseling assembly comprises a knife edge and a knife rod;
the knife edge is connected with the knife rod, the knife edge and the knife rod are slidably provided in the guide pipe, the knife rod abuts against the elastic member, and one end of the knife rod away from the knife edge is connected with the rotation shaft of the motor via the pull string.

14. The method according to claim 13, wherein a limiting portion is provided on the knife rod, a limiting groove is provided on a side wall of the guide pipe, and the limiting portion is slidably provided in the limiting groove.

15. The method according to claim 11, wherein the detasseling device further comprises a main beam;
at least one side of the main beam is provided with a slid rail, and the detasseling support of the detasseling module is slidably provided on the slid rail.

16. The method according to claim 11, wherein a gyroscope and a damping module are provided in the moving device, the gyroscope is configured for detecting an inclination degree of the moving device, and the damping module is configured for adjusting an inclination angle of the moving device based on the inclination degree.
